(19) United States
(12) Patent Application Publication
(10) Pub. No.: US 2026/0061080 A1
(43) Pub. Date: Mar. 5, 2026

(54) FLUORESCENT COMPOUND BASED ON NEW INDOCYANINE GREEN IR820, AND PREPARATION AND USE THEREOF

(71) Applicant: Shanghai Fluoresoma Medical Technology Co., Ltd., Shanghai (CN)

(72) Inventors: Chusen HUANG, Shanghai (CN); Jigao XUAN, Shanghai (CN); Nengqin JIA, Shanghai (CN)

(73) Assignee: Shanghai Fluoresoma Medical Technology Co., Ltd., Shanghai (CN)

(21) Appl. No.: 19/382,210

(22) Filed: Nov. 6, 2025

Related U.S. Application Data
(63) Continuation of application No. PCT/CN2024/101326, filed on Jun. 25, 2024.

(30) Foreign Application Priority Data
Jul. 5, 2023 (CN) 202310818545.2

(51) Int. Cl. A61K 49/00; C07K 1/107

(52) U.S. Cl. CPC A61K 49/0056; A61K 49/0034; C07K 1/1077

(57) ABSTRACT

The present invention relates to a fluorescent compound based on new indocyanine green IR820, and preparation and use thereof. The fluorescent compound has a molecular structural formula of formula (1):

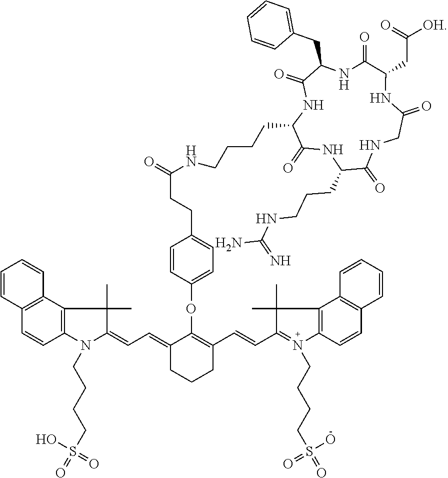

Compared with the prior art, a fluorescent probe of the present invention has near-infrared fluorescence emission, good tumor targetability, biocompatibility, low cytotoxicity, and strong biological tissue penetrability, can be used for targeted fluorescence imaging of tumor cells and living tumors, and is expected to be further used in fluorescence-guided surgery.

### CROSS-REFERENCE TO RELATED APPLICATION

This application is a continuation of international application of PCT application serial no. PCT/CN2024/101326, filed on Jun. 25, 2024, which claims the priority benefit of China application no. 202310818545.2, filed on Jul. 5, 2023. The entirety of each of the above-mentioned patent applications is hereby incorporated by reference herein and made a part of this specification.

### TECHNICAL FIELD

The present invention relates to the technical field of fluorescent compounds, and relates to a fluorescent compound based on new indocyanine green IR820, and preparation and use thereof.

### BACKGROUND OF RELATED ART

Fluorescence imaging has exhibited a broad application prospect in clinical transformation aspects, such as basic research in biomedicine and accurate intraoperative resection of tumors. One of most core technologies for the fluorescence imaging is to create fluorescent probe molecules capable of being used for imaging. The fluorescent probe molecules can dynamically track various physiological and pathological processes and occurrence and progression of diseases at a molecular level. Particularly, in the field of tumor visualization imaging, since the fluorescent probe molecules can brighten cancer cells in real time during surgery, physicians can be assisted in determining tumor boundaries and identifying metastatic lesions more accurately. Although there are a large number of reports on fluorescent probes for tumor imaging in current academic papers, few probe molecules have been actually approved by the FDA for clinical tumor imaging currently, mainly because biotoxicity of most fluorescent dyes used for constructing the fluorescent probes currently leads to limited clinical use. Up to now, the fluorescent dyes approved by the FDA for clinical use only include fluorescein, methylene blue, and indocyanine green (ICG). However, due to fluorescence emission wavelengths limited within 700 nm, the fluorescein and the methylene blue are easily interfered with endogenous background fluorescence signals of organisms when used for tumor fluorescence imaging, and also have lower biological tissue penetrability. Therefore, the fluorescent probes developed based on the fluorescein and the methylene blue are only suitable for use in laparotomy, and are easily interfered by the background fluorescence signals of the organisms. The ICG, due to an emission wavelength of approximately 800 nm, is currently the only fluorescent dye approved by the FDA for fluorescence-guided surgery. Therefore, development of the fluorescent probe molecules based on the ICG dye has always been a focus point in the tumor fluorescence imaging. However, the ICG has two major problems when used for the tumor fluorescence imaging in the organisms. (1) The ICG does not have targetability and requires either intratumoral injection or relies on passive targeting (with an enhanced permeability and retention effect (EPR) in a tumor region) for accumulation in the tumor region after injection, and high-contrast tumor fluorescence imaging can only be achieved after metabolism of a free dye. As a result, operational complexity is caused during the tumor imaging. (2) The ICG dye is prone to photobleaching after long-term excitation. Thus, the ICG has insufficient photostability and cannot be excited for a long time during the tumor fluorescence imaging, leading to limited use to a certain extent.

To address the above problems, according to current reports, a tumor-targeting group is directly linked to a side chain of the ICG to solve a tumor targeting problem. On the other hand, a stable six-membered ring structure introduced onto a long-chain conjugated portion of the ICG can greatly improve the photostability of the dye, thereby obtaining a series of ICG derivative dyes, such as IRDye800CW and ZW800-1 dyes. However, since two sulfonic acid groups are introduced onto a benzene ring of the IRDye800CW dye and two positively charged quaternary ammonium salts are introduced onto a side chain of the ZW800-1 dye, the dyes are greatly different from an ICG structure in charge method (the ICG carries two negative charges on the side chain). Therefore, the dyes may be changed to a certain extent in terms of biocompatibility, but require further clinical validation, and thus cannot be compared with the ICG, which has been approved by the FDA for use for a long time and has been validated to have good biocompatibility. Further, different charge methods of the dyes directly affect magnitude of background signals (mainly derived from nonspecific adsorption to biomacromolecules). IR820, also known as new indocyanine green, structurally and completely retains the charge method and mother nucleus structure of the ICG in terms of charge method and chemical structure, compared with other ICG-based analog fluorescent dyes, such as the IRDye800CW and the ZW800-1. Therefore, in terms of biocompatibility, it is most similar to the ICG, which has been approved by the FDA and has been clinically validated to have better biocompatibility for a long time. Currently, many tumor-targeted fluorescent probes have been designed based on the IR820. However, these probes are synthesized based on an encapsulating effect of a nanomaterial on the IR820. Although these nanoprobes synthesized based on the IR820 have exhibited certain potential in terms of tumor imaging, the nanoprobes may have greater renal and hepatic toxicity in terms of biological metabolism compared with organic small-molecule probes, since the nanoprobes have larger dimensions (nanoscale). There are few tumor-targeted labeled small-molecule fluorescent probes based on the IR820 dye, and currently, there are no organic small-molecule fluorescent probes based on the IR820 designed for integrin avβ3 highly expressed on surfaces of tumor cells. On the other hand, in coupling of a targeting group with the IR820, a meso-chloro substituent of the IR820 is the only group available for coupling. However, during the coupling, the meso-chloro group of the IR820 is easily substituted by groups such as amino and thio (Sci. China Chem. 2020, 63, 699-706). Thus, the coupling reaction has many side reactions, leading to a greater challenge in synthesis of a target probe by directly introducing the targeting group through a substitution reaction of the meso-chloro of the IR820. Meanwhile, during subsequent coupling reactions utilizing the IR820, since the IR820 has two sulfonic acid groups, purification has a certain difficulty. Therefore, a coupling product in each step is typically separated by adopting reverse-phase high performance liquid chromatography (HPLC), which requires large reverse-phase preparative HPLC equipment and consumes a large number of solvents and a lot of time. Exploring simple and convenient novel synthetic processes is beneficial for reducing costs of fluorescent reagents developed based on the IR820, and also lays a foundation for marketization.

Methods for preparing IR820-based cRGD cyclic peptides for targeting by introducing integrin avβ3 highly expressed on surfaces of tumor cells have also not been reported yet. Meanwhile, although the emission wavelengths of the IRDye800CW and the ZW800-1 are approximately 800 nm, reaching the near-infrared region, they are approximately 20 nm shorter than an emission wavelength (820 nm) of the new indocyanine green dye (IR820). Therefore, bevacizumab-IRDye800CW and cRGD-ZW800-1 developed based on the above two dyes have lower biological tissue penetrability than the IR820.

### SUMMARY OF THE INVENTION

An objective of the present invention is to provide a fluorescent compound based on new indocyanine green IR820, and preparation and use thereof.

The objective of the present invention may be achieved through the following technical solutions.

A first technical solution of the present invention provides a fluorescent compound based on new indocyanine green IR820, which has a molecular structural formula of formula (1):

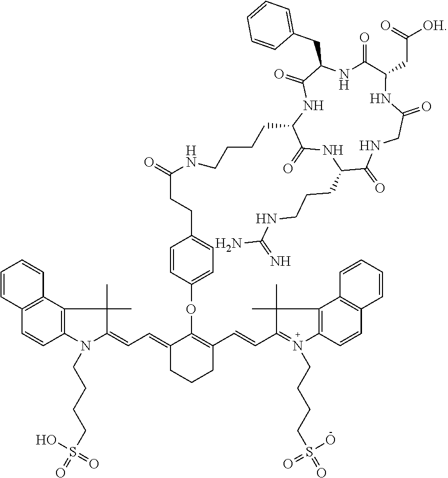

A second technical solution of the present invention provides a method for preparing the fluorescent compound based on new indocyanine green IR820, which includes enabling a compound of formula (2) to sequentially react with a precursor of a linker arm and cRGD to obtain a target product;

where, the compound of formula (2) has a molecular structural formula as follows:

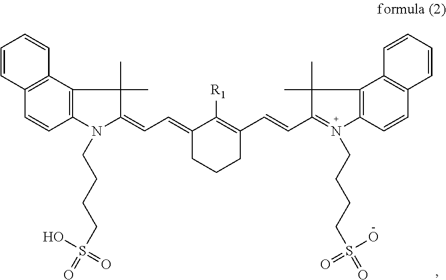

where R₁ is selected from a halogen element; and

the precursor of the linker arm includes a compound having a structure of formula (3):

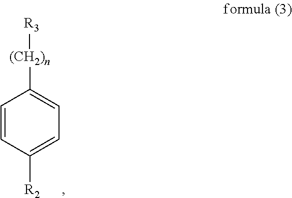

where R₂ includes a hydroxyl, R₃ includes a carboxyl, and n is 1-10.

Further, R₁ is Cl.

Further, R₂ is the hydroxyl, R₃ is the carboxyl, and n=2.

Further, a molar ratio of the compound of formula (2), the precursor of the linker arm, and the cRGD is 1:(1-10):(1-3), and may be specifically 1:1:1, 1:10:3, 1:5:2, or any intermediate point value within this range.

Further, the compound of formula (2) sequentially reacts with the precursor of the linker arm in an organic solvent system (for example, dimethylformamide, DMF), sodium hydride is further added into the reaction system, a reaction temperature is room temperature, and a reaction time is 3-5 h.

Further, an intermediate product obtained after the compound of formula (2) reacts with the precursor of the linker arm is first subjected to activation treatment with ethyldimethylaminopropyl carbodiimide (EDC) and N-hydroxysuccinimide (NHS) in an N-methylpyrrolidone (NMP) or DMF solvent, and then reacts with the cRGD at a reaction temperature of room temperature.

A third technical solution of the present invention provides use of the fluorescent compound based on new indocyanine green IR820 in preparation of a fluorescence-guided surgery probe.

A fourth technical solution of the present invention provides a fluorescent composition, which includes the above-mentioned fluorescent compound based on new indocyanine green IR820, and a pharmaceutically acceptable carrier.

A fifth technical solution of the present invention provides a fluorescence imaging system, which includes a fluorescence detection device and a fluorescent probe, where the fluorescent probe includes the above-mentioned fluorescent compound based on new indocyanine green IR820.

In addition, the present invention further provides a fluorescence imaging method based on non-diagnostic and non-therapeutic purposes, which includes: administering a fluorescent probe to a subject, followed by fluorescence imaging of the subject; where the subject includes living cells, active physiological tissues of animals, or living animals; and the fluorescent probe includes the fluorescent compound.

Compared with the prior art, the present invention has the following advantages.
(1) A tumor-targeted fluorescent probe based on a mother nucleus structure of ICG is provided, and compared with the ICG approved by the FDA, the fluorescent probe of the present invention has better tumor targetability and good photostability.
(2) Compared with currently reported fluorescent probes, such as bevacizumab-IRDye800CW and cRGD-ZW800-1, developed based on ICG analogs such as IRDye800CW and ZW800-1, the fluorescent probe of the present invention completely retains the charge method and mother nucleus structure of the ICG in charge method and chemical structure. Therefore, in terms of biocompatibility, it is most similar to the ICG, which has been approved by the FDA and has been clinically validated to have better biocompatibility for a long time.
(3) Currently, there are no tumor-targeted organic small-molecule fluorescent probes based on the IR820 fluorescent dye and using the cRGD as a targeting group. The present application provides a novel tumor-targeted organic small-molecule fluorescent probe based on the IR820 fluorescent dye.
(4) In the present invention, the linker arm with phenolic hydroxyl is first introduced onto meso-chloro of the IR820, and then the linker arm is further coupled with a desired targeting group. In this way, the phenolic hydroxyl is used to first substitute the chloro on the IR820 to form stable phenol-substituted IR820, thereby avoiding unsuccessful coupling synthesis caused by easy impacts of coupling auxiliary agents, such as amino or thio, on the chlorine atom during direct coupling of the IR820 with the targeting group. Therefore, the present application provides a synthetic method for successfully coupling the IR820 with a targeting group.
(5) In the present invention, after the IR820 is currently coupled with the group, products are purified and obtained by reverse-phase preparative HPLC in most reactions in each step. In the present invention, a pure product of IR820-COOH is obtained by column chromatography (with a developing agent including dichloromethane and methanol at 10:1-5:1), thereby reducing loss and consumption of instruments, equipment, and labor power, and achieving a yield close to that of reverse-phase preparative HPLC.

### BRIEF DESCRIPTION OF THE DRAWINGS

FIG. 1 is a mass spectrogram of IR820-COOH in an example of the present invention.

FIG. 2 is a mass spectrogram of IR820-cRGD in an example of the present invention.

FIG. 3 is an HPLC diagram of the IR820-cRGD in an example of the present invention.

FIG. 4 shows fluorescence and bright field photos of the IR820-cRGD in an example of the present invention with different cells.

FIG. 5 shows in vivo fluorescence imaging images of the IR820-cRGD in an example of the present invention in mouse tumors.

### DETAILED DESCRIPTION OF THE EMBODIMENTS

The present invention is described in detail below in combination with the accompanying drawings and specific examples. The examples are implemented based on the technical solutions of the present invention, and detailed embodiments and specific operation processes are provided, but the scope of protection of the present invention is not limited to the following examples.

In the following examples, structural formulas of mentioned ICG, IR820, IRDye800CW, and ZW800-1 are as follows:

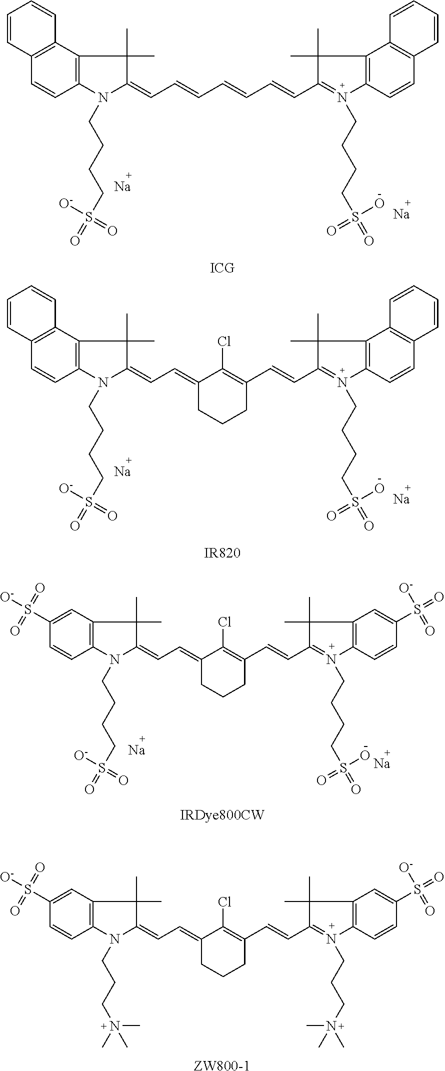

cRGD was purchased from Xi'an Qiyue Biotechnology Co., Ltd.

Unless otherwise specified, it is indicated that all other raw materials or treatment techniques are conventional commercially available raw materials or conventional treatment techniques in the art.

### Example 1

A fluorescent compound provided in this example may be named as IR820-cRGD, which has a synthetic route as follows:

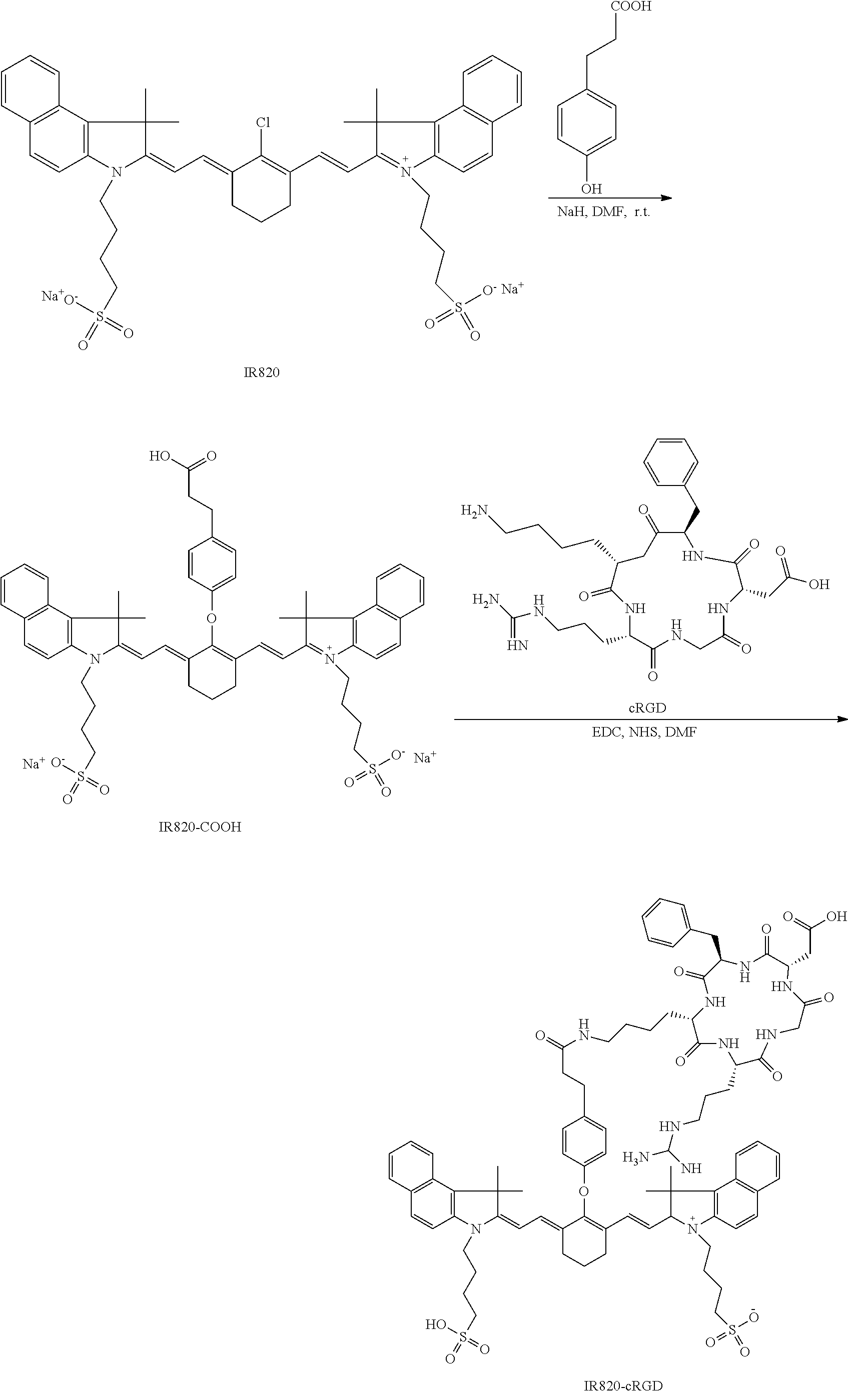

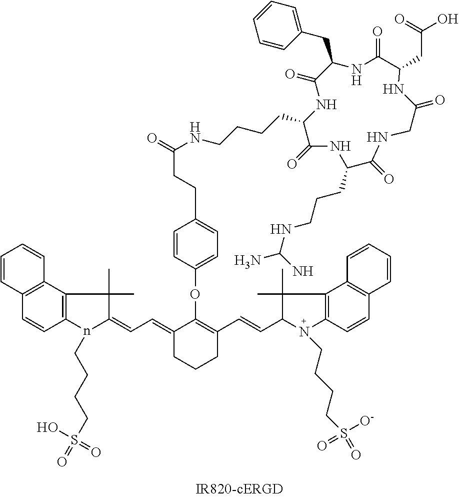

Specifically, a synthetic method for the fluorescent compound includes the following steps.

(1) Synthesis of intermediate IR820-COOH:

332 mg of p-hydroxyphenylpropionic acid was dissolved in 10 ml of anhydrous N,N-dimethylformamide (DMF), 47 mg of sodium hydride was added, and stirring was performed under the protection of nitrogen at room temperature for half an hour. 170 mg of IR820 was added, and stirring was performed continuously for a reaction at room temperature for 4 hours. Thin-layer chromatography (TLC) showed that the raw material IR820 disappeared, and a new spot appeared. The reaction was terminated, and the reaction solution was added into methyl tert-butyl ether until a large amount of a blackish green solid appeared. Centrifugation was performed to obtain the solid. The solid was washed with 10 ml of acetone or ethyl acetate for three times. The obtained solid was dried by vacuum drying to obtain a crude product. The crude product was further subjected to column chromatography (with a developing agent including dichloromethane and methanol at 10:1-5:1) to obtain a pure product with a metallic luster and a light green color, with a yield of 32%.

¹H NMR (400 MHZ, DMSO-d₆) δ 8.87 (d, J=14.1 Hz, 2H), 8.26 (d, J=8.5 Hz, 2H), 8.07-8.03 (m, 4H), 7.75 (d, J=9.0 Hz, 2H), 7.63 (t, 8.5, 2H), 7.49 (t, J=7.5 Hz, 2H), 7.11 (d, J=8.6 Hz, 2H), 6.59 (d, J=8.5 Hz, 2H), 6.37 (d, J=14.2 Hz, 2H), 4.30 (s, 4H), 3.53 (s, 2H), 2.75 (d, J=5.9 Hz, 4H), 2.63 (d, J=8.8 Hz, 4H), 2.58 (t, J=7.3 Hz, 4H), 2.10-1.66 (m, 22H). 13C NMR (101 MHZ, DMSO-d6) δ 172.24, 151.56, 146.96, 144.03, 139.17, 132.82, 132.66, 130.69, 129.75, 129.25, 128.06, 127.08, 126.87, 124.23, 121.65, 120.46, 114.47, 111.10, 100.52, 54.25, 50.07, 49.87, 43.12, 39.43, 39.38, 39.22, 39.17, 39.01, 38.96, 38.80, 38.76, 38.55, 38.34, 38.13, 26.35, 25.69, 25.26, 21.80, 21.12.

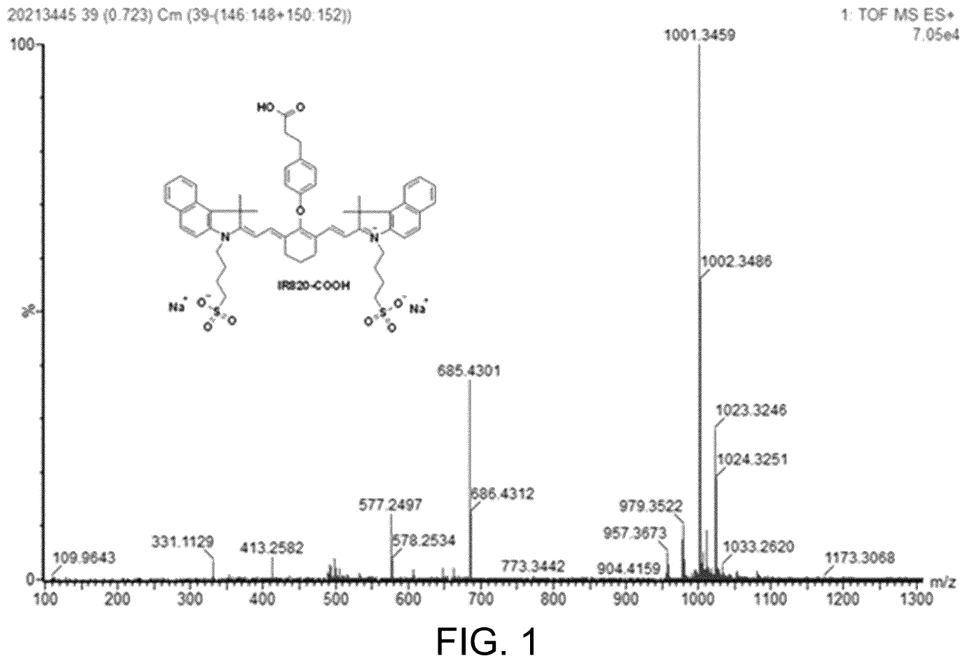

HRMS (ES+, m/z): calcd for C55H59N209S2+: 1001.3452, found: 1001-3459. A mass spectrogram refers to FIG. 1.

### (2) Synthesis of IR820-cRGD:

100 mg of the IR820-COOH was dissolved in NMP or DMF, 10 times equivalent amounts of EDC and NHS were added for activation for half an hour, then an equal equivalent amount of cRGD was added, and stirring was performed continuously for a reaction at room temperature overnight. TLC showed that the raw material IR820-COOH disappeared, and a new spot appeared. The reaction was completed. Separation was performed by HPLC to obtain a green liquid, which was then freeze-dried to obtain a grass green solid, namely the target product IR820-cRGD, with a yield of approximately 20%.

¹H NMR (400 MHZ, DMSO-d₆) δ 8.15 (d, J-8.4 Hz, 2H), 8.04-7.95 (m, 5H), 7.74 (d, J=8.8 Hz), 7.62-7.58 (m, 6H), 7.49-7.45 (m, 2H), 7.28 (d, J-8.0 Hz, 2H), 7.16 (d, J=8.0 Hz, 2H), 6.71 (s, br, 1H), 6.26 (d, J=12.4 Hz, 2H), 4.27 (s, 4H), 4.25-4.17 (m, 2H), 4.12-4.05 (q, J1=7.5 Hz, J2=13.5 Hz, 2H), 3.92 (s, 4H), 3.89-3.82 (q, J1=6.0 Hz, J2=15.0 Hz, 2H), 3.73-3.65 (m, 4H), 3.13 (s, 6H), 3.04 (s, 2H), 2.77 (s, 4H), 2.65-2.64 (m, 4H), 2.46-2.45 (m, 6H), 2.35-2.21 (m, 4H), 2.10-2.06 (m, 8H), 1.96-1.80 (m, 6H), 1.88 (d, J-6.0 Hz, 1H), 1.40 (s, 2H), 1.07-0.99 (m, 6H). 1.22 (s, 12H).

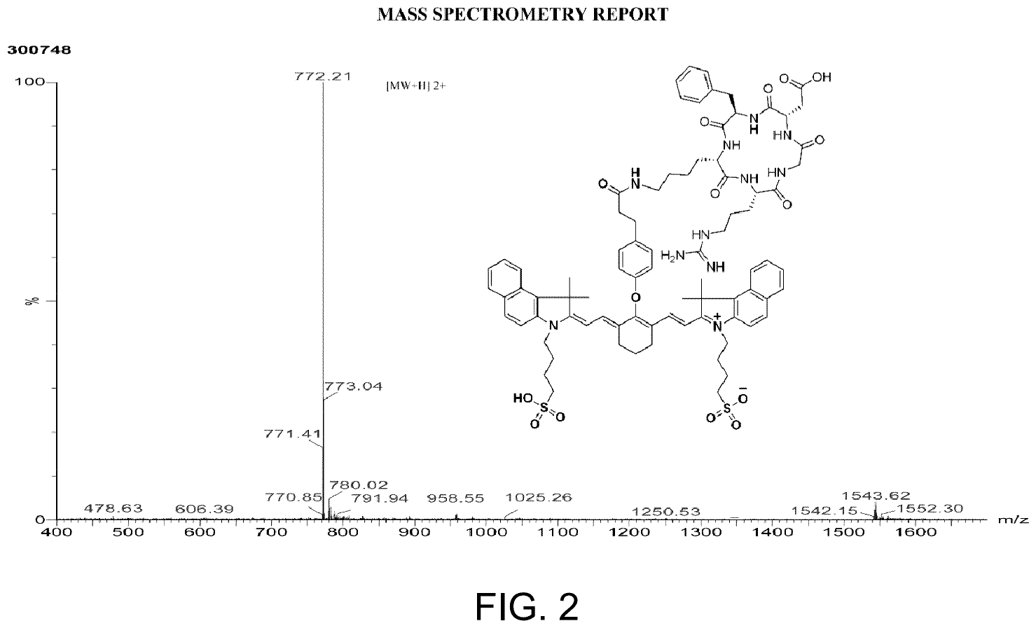

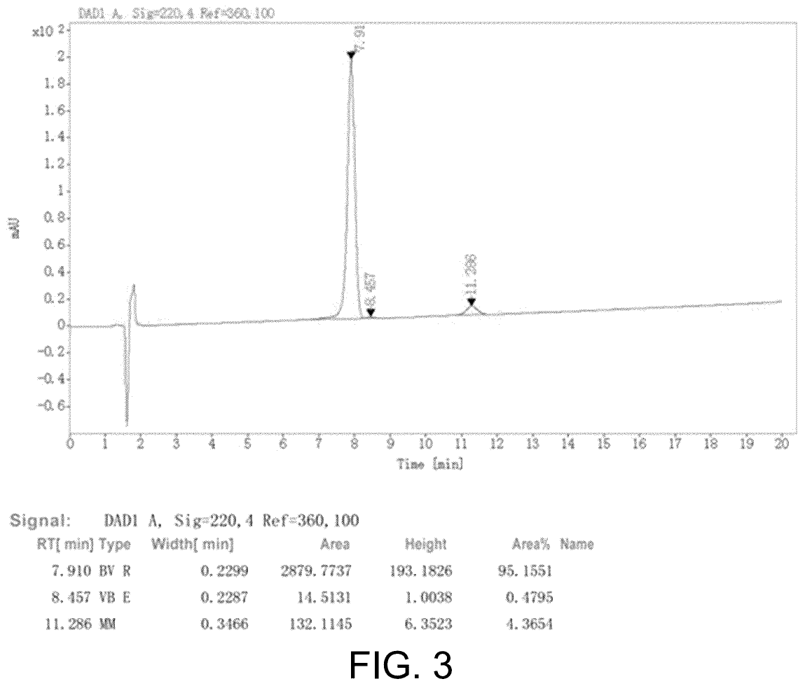

LC-MS (ES+, m/z): [M+H]²⁺, 772.21. The product was determined by HPLC to obtain a purity of approximately 95% (buffer solution A: 0.1% TFA in H2O; and buffer solution B: 0.1% TFA in acetonitrile). A mass spectrogram refers to FIG. 2, and HPLC is shown in FIG. 3.

### Example 2

### Fluorescence Imaging of IR820-cRGD in Living Cells

An instrument model used for cell imaging is a Leica TCS SP5 II confocal laser scanning microscope using a HC×PLAPO 63×oil objective (NA: 1.40).

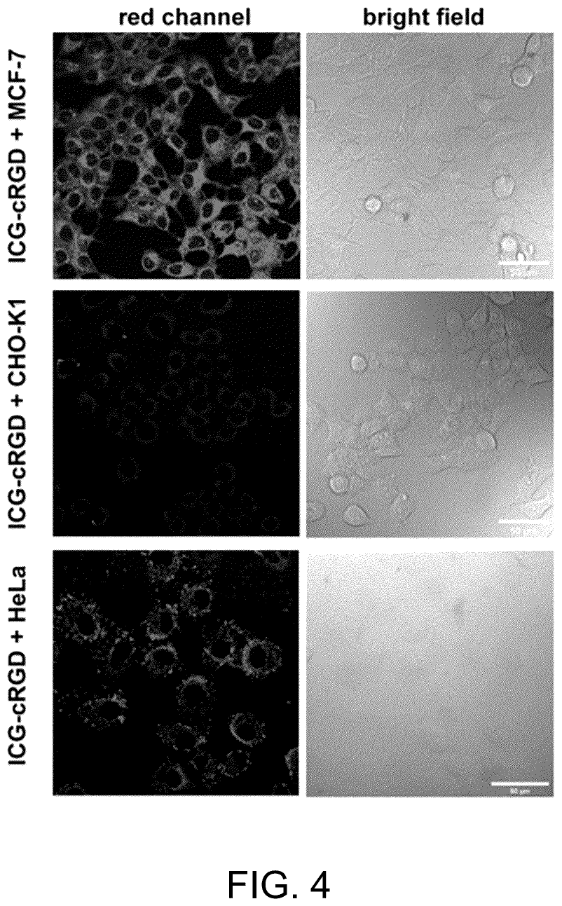

The experiment was carried out in three groups, with one group using normal cells (CHO), one group using breast cancer cells (MCF-7), and one group using cervical cancer cells (Hela). Before an imaging test, a cultured cell culture solution was first sucked away, and the cells were washed once with a PBS buffer solution, and then washed once with DMEM or 1640. 10 μL of a formulated DMSO mother solution with a probe concentration of 1 mM was measured and added into a 2 mL culture dish containing a fresh DMEM or 1640 culture medium. After culture was completed, the excessive culture solution was first removed, the cells were washed with a PBS buffer solution (pH 7.4) to remove an excessive probe, and then the imaging test was carried out on the cells separately using a confocal fluorescence microscope. FIG. 4 shows fluorescence imaging images of the probe in the three cell types. It can be shown that the probe has greater selective fluorescence signals for the tumor cells. It is shown that the probe has potential for tumor imaging.

### Example 3

### Fluorescence Imaging of IR820-cRGD in Living Mice

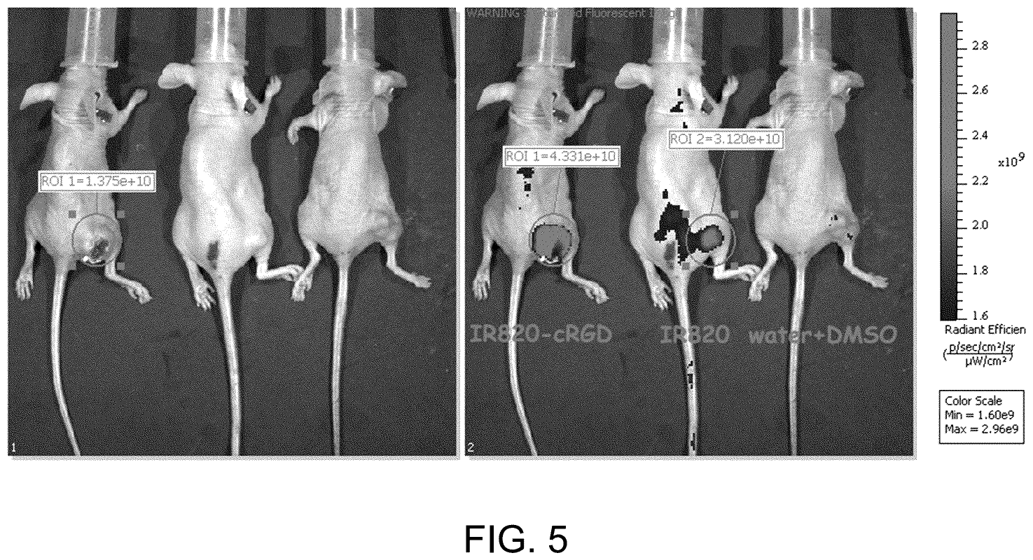

Three tumor-bearing mice were administered with an IR820-cRGD solution in pure water at a concentration of 10 μM, a DMSO solution in pure water at an equal equivalent probe concentration, and an IR820 solution in pure water at an equal equivalent probe concentration through tail vein injection, respectively. 2 hours later, tumor sites of the mice were observed using an in vivo imaging instrument for small animals to obtain imaging situations. As shown in FIG. 5, the IR820-cRGD shows a highly bright fluorescence signal at the tumor site of the mouse. On the contrary, the DMSO solution in pure water shows no fluorescence signal, and the non-targeted IR820 only shows a weak fluorescence signal. The results indicate that the target probe IR820-cRGD greatly improves an imaging effect of the IR820 dye for living tumors.

### Example 4

Compared with Example 1, most conditions are the same, except that a molar ratio of the IR820, the p-hydroxyphenylpropionic acid, and the cRGD is adjusted to 1:1:1.

### Example 5

Compared with Example 1, most conditions are the same, except that a molar ratio of the IR820, the p-hydroxyphenylpropionic acid, and the cRGD is adjusted to 1:10:3.

The above description of the examples is to facilitate understanding and use of the present invention by those of ordinary skill in the art. Obviously, those skilled in the art can easily make various modifications to these examples and apply general principles described herein to other examples without creative labor. Therefore, the present invention is not limited to the above examples, and all improvements and modifications that are made by those skilled in the art according to the revelation of the present invention without departing from the scope of the present invention shall fall within the scope of protection of the present invention.

What is claimed is:

1. A fluorescent compound based on new indocyanine green IR820, having a molecular structural formula of formula (1):

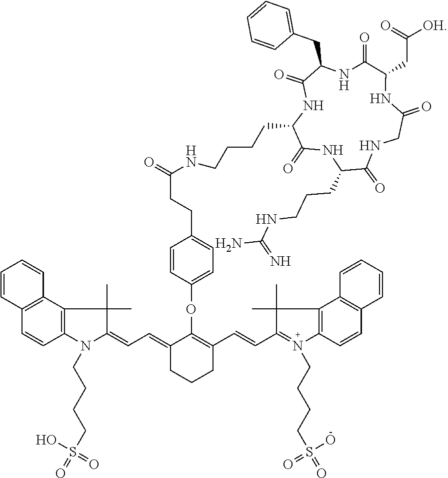

2. A method for preparing the fluorescent compound based on the new indocyanine green IR820 according to claim 1, comprising enabling a compound of formula (2) to sequentially react with a precursor of a linker arm and cRGD to obtain a target product;
wherein, the compound of formula (2) has a molecular structural formula as follows: 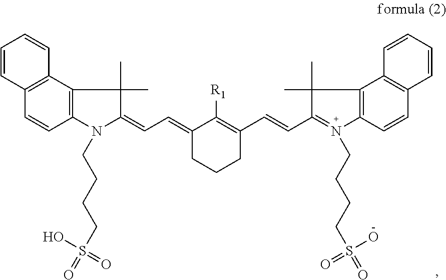
wherein R₁ is selected from a halogen element; and
the precursor of the linker arm comprises a compound having a structure of formula (3): 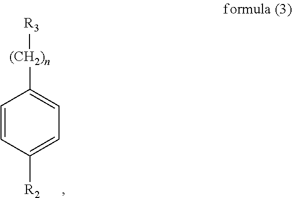
wherein R₂ comprises a hydroxyl, R₃ comprises a carboxyl, and n is 1-10.

3. The method for preparing the fluorescent compound based on the new indocyanine green IR820 according to claim 2, wherein R₁ is Cl.

4. The method for preparing the fluorescent compound based on the new indocyanine green IR820 according to claim 2, wherein R₂ is the hydroxyl, R₃ is the carboxyl, and n=2.

5. The method for preparing the fluorescent compound based on the new indocyanine green IR820 according to claim 2, wherein a molar ratio of the compound of formula (2), the precursor of the linker arm, and the cRGD is 1:(1-10):(1-3).

6. The method for preparing the fluorescent compound based on the new indocyanine green IR820 according to claim 2, wherein the compound of formula (2) sequentially reacts with the precursor of the linker arm in an organic solvent system, sodium hydride is further added into the reaction system, a reaction temperature is room temperature, and a reaction time is 3-5 h.

7. The method for preparing the fluorescent compound based on the new indocyanine green IR820 according to claim 2, wherein an intermediate product obtained after the compound of formula (2) reacts with the precursor of the linker arm is first subjected to activation treatment with ethyldimethylaminopropyl carbodiimide (EDC) and N-hydroxysuccinimide (NHS) in an N-methylpyrrolidone (NMP) or dimethylformamide (DMF) solvent, and then reacts with the cRGD at a reaction temperature of room temperature.

8. Use of the fluorescent compound based on the new indocyanine green IR820 according to claim 1 in preparation of a fluorescence-guided surgery probe.

9. A fluorescent composition, comprising the fluorescent compound based on the new indocyanine green IR820 according to claim 1, and a pharmaceutically acceptable carrier.

10. A fluorescence imaging system, comprising a fluorescence detection device and a fluorescence probe, wherein the fluorescence probe comprises the fluorescent compound based on the new indocyanine green IR820 according to claim 1.
